(19) United States
(12) Patent Application Publication
(10) Pub. No.: US 2023/0340392 A1
(43) Pub. Date: Oct. 26, 2023

(54) CELL DEPOSITION AND IMAGING APPARATUS

(71) Applicant: FFEI LIMITED, Waterbeach, Cambridge (GB)

(72) Inventor: Richard Michael SALMON, Leighton Buzzard (GB)

(73) Assignee: FFEI LIMITED, Waterbeach, Cambridge (GB)

(21) Appl. No.: 18/028,971

(22) PCT Filed: Sep. 27, 2021
(86) PCT No.: PCT/GB21/52508

§ 371 Date: Mar. 28, 2023

(30) Foreign Application Priority Data
Sep. 29, 2020 (GB) 2015366.4

(51) Int. Cl. C12M 1/34; G01N 21/35; C12M 1/00; C12M 1/36

(52) U.S. Cl. CPC C12M 41/36; G01N 21/35; C12M 41/14; C12M 41/48

(57) ABSTRACT

A cell deposition and imaging apparatus comprises: a printing mechanism comprising at least one channel, the at least one channel of the printing mechanism arranged to: receive a sample of a cell-carrying fluid comprising at least one cell-type; and deposit the sample of the cell-carrying fluid onto a target area of a substrate, an imaging system arranged to image the target area; and a transportation system arranged to move the target area between a printing position, in which the target area is located substantially adjacent to the printing mechanism, and an imaging position, in which the target area is located substantially adjacent to the imaging system; wherein the imaging system comprises an imager capable of imaging a region of the substrate wherein the region is smaller than the target area and the imaging system is arranged to image all of the target area by moving the target area relative to the imager.

### FIELD OF INVENTION

The invention relates to a cell deposition and imaging apparatus for depositing and scanning biological material.

### BACKGROUND

Mechanical and optical technologies are currently used to create bright field digital pathology slide scanners for medical imaging and digital printing engines.

Multi-drop microarray technology (biochips, genome sequencing) assessed with fluorescent/chromogenic signals and digital bright field imaging (digital microscopes, confocal microscopes, WSI scanners) of low copy number samples currently exist as commercial products in separate independent forms.

Modern industrial Life Science research focuses on high-throughput methodologies to rapidly and reliably discover, develop and manufacture therapeutics. Most life science processes target biomolecules (DNA and proteins) and cells in solution. The highest throughput and statistical relevance is obtained by having thousands of small-scale experiments of different targets analysed in multiplicate in the same place at the same time.

Current technology uses independent processes with single-function technologies to perform biological analysis. Secondary markers of fluorescence and colourimetry often give relative quantitation for protein/DNA microarrays and single-cell technology, inferring outcomes via signal interpretation with little in the way of direct, real-time observation. The exact morphological changes a drug/biomolecule has on living cells is rarely recorded in high-throughput methods, and are commonly restricted to single-sample investigations.

Visualising methods such as digital or manual microscopes classically accept individual or low copy-number samples, such as microscope slides or 6-24-well trays, and in doing so measure relative samples one-at-a-time. The result is either independent samples in a living experiment suffering from variation in time, thus requiring more extensive statistical repeats for consensus, or fixed/dead samples to eliminate time variation but preventing onward study.

Current technology that addresses eliminating time as a sample-to-sample variable does so by including one sensor/sample, therefore massively increasing cost proportionally to throughput. As these drop-array and detection processes are commonly disparate and performed with several devices, laboratory workflows are sub-optimal and require technicians to be trained to operate a multitude of machines, often simultaneously.

### SUMMARY

Aspects and embodiments of the present disclosure provide a cell deposition apparatus as defined in the appended claims.

According to a first aspect of the invention there is provided a cell deposition and imaging apparatus comprising: a printing mechanism comprising at least one channel, the at least one channel of the printing mechanism arranged to: receive a sample of a cell-carrying fluid comprising at least one cell-type; and deposit the sample of the cell-carrying fluid onto a target area of a substrate, an imaging system arranged to image the target area; and a transportation system arranged to move the target area between a printing position, in which the target area is located substantially opposite the printing mechanism, and an imaging position, in which the target area is located substantially opposite the imaging system; wherein the imaging system comprises an imager capable of imaging a region of the substrate wherein the region is smaller than the target area and the imaging system is arranged to image all of the target area by moving the target area relative to the imager.

Preferably the region of the substrate being imaged by the imager is substantially smaller than the target area, and further preferably, the imaging system uses at least one light source compatible with imaging the samples deposited in the target area of the substrate.

The cell deposition apparatus therefore provides a complete “lab-in-a-box” system that is able to prepare complex biological experiments by depositing multiple samples using a high-precision printing mechanism and image the resulting deposition, without the need for user interaction throughout the duration of the experiment. The cell deposition apparatus therefore provides a complete, automated experimental system.

The printing mechanism may be arranged to receive multiple samples of cell-, biomolecule- or microparticle-carrying fluid (hereby all referred to as ‘cell-carrying fluid’) and deposit the multiple samples of the cell-carrying fluid onto a target area of a substrate. The samples may be deposited in the form of drops onto the target area. These drops may be discretely deposited so that each drop is separate and distinct from any other drop. Separate drops allow the volume of fluid in each drop to be selected prior to printing so that the relative quantities of fluid in each drop can be selected depending on the user's experiment.

In other examples, the samples may be deposited in the form of 1 drop or 2 or more connected drops to form an elongated drop, having an extended elliptical shape. In other examples, the input fluid, with or without cells, may be capable of gelling or solidifying post-deposition in order to customise the surface of the substrate with biocompatible structures.

In some examples, the printing mechanism may be arranged to receive and deposit solutions such as those used to prepare or coat the substrate surface to prevent drop spreading (such as siliconization), introduce nutrients that support biology (such as like growth medium or agar in a petri dish) or solutions that gel or solidify to customise and structure the surface of the substrate.

The imaging system may be arranged to image the multiple samples in the target area substantially simultaneously. In this case, substantially simultaneously means that the time at which the first sample is imaged is substantially the same as the time at which the last sample is imaged. For example, the time difference between the first and last sample being imaged and collected within a 15 mm×15 mm area is preferably less than a minute.

The printing mechanism may comprise a plurality of individual printheads arranged as an array of printheads. The individual printheads may be arranged in a 2-dimensional array, comprising n×m individual printheads. This allows multiple samples of cell-carrying fluid to be deposited in one go in multiple different position of the target area in one printing action. This allows for quicker, more efficient printing of the cell-carrying fluid onto the target area of the substrate, which is important when very large numbers of drops are required to be printed. Alternatively, the individual printheads may be arranged in a 1-dimensional array, comprising n individual printheads.

The plurality of individual printheads in the array of printheads may be arranged to move together as a single unit such that there is no relative movement between individual printheads within the array. This ensures that there is a constant spacing between each individual printhead, and consequently between each deposited drop, on the target area. The drops on the target area are therefore regularly and equally spaced across the target area on the substrate. This also reduces the number of mechanisms required to move during operation of the apparatus.

In some examples, the individual printheads within the array of printhead are able to move relative to each other within the array. For example, the plurality of printheads may be arranged to independently move relative to each other. This allows the spacing between individual printheads, and consequently the spacing between each deposited drop, to vary on the target area. This may be advantageous when the drops deposited from each printhead are not equal in size and so the spacing between different drops needs to be varied, depending on the size on the drop.

Each individual printhead may comprise a channel arranged to receive and transfer the cell-carrying fluid through the printing mechanism and onto the substrate. In some instances, the receiving channel may be connected to the output from a free-standing or integrated cell-sorting and cell-identifying device such as but not limited to a fluorescent-activated cell-sorter (FACS), magnetic-activated cell sorter (MACS) or flow cytometer.

Each channel of the printing mechanism may be arranged to receive a respective cell-carrying fluid to be deposited on a target area of the substrate. The cell-carrying fluid typically comprises a carrier fluid and at least one cell. In some cases the cell-carrying fluid comprises at least one cell, biomolecule, or non-biological microparticle. However, in other cases, the cell-carrying fluid does not include any cells but only contains carrier fluid and optionally a non-cell biomolecule (for example a protein/antibody/enzyme, nucleic acid, drug, antibiotic, reporter chemical, inhibitor etc). The respective cell-carrying fluids received by each of the channels in the printing mechanism may differ in their respective compositions. For example each cell-carrying fluid may include a different cell and/or a different carrier fluid. This may allow different experiments to be carried out and compared on a single substrate. For example, the effects of different drugs on the same cell can be investigated or the effects of the same drug on different cells can also be investigated.

In another embodiment, a fluid containing non-biological cell-mimicking microparticles may be used to simulate a biological experiment or for the purposes of manufacturing and calibration processes.

For some experiments, it may be advantageous to combine the multiple samples of cell-carrying fluid within the target area of the substrate. The printing mechanism may therefore be able to deposit multiple samples of cell-carrying fluid on the same part of the target area.

Each channel in the array of printheads may receive cell-carrying fluid that has been pre-processed by a free-standing or integrated cell-sorter such as a flow cytometer, fluorescence-activated cell sorter (FACS) or a magnetic-activated cell sorter (MACS).

The printing mechanism may be arranged to overprint pre-deposited unstained or unlabelled biological experiments with cell-staining or labelling solutions substantially immediately prior to transportation to the imaging system.

In order to allow the printing mechanism to be moved relative to the transportation system, the printing mechanism may be arranged to be mounted on a carrier mechanism. In particular, the carrier mechanism may allow the printing mechanism to be moved relative to the target area. The carrier mechanism may include a track along which the printing mechanism moves. The movement may be lateral movement, typically a shuttling, back-and-forth movement, confined to the horizontal plane. The track may allow the printing mechanism to move in the x-direction and the y-direction within the horizontal plane. Advantageously, moving the printing mechanism relative to the transportation system, and in particular moving it relative to the target area, allows the printing mechanism to be positioned over different parts of the target area so that the printing mechanism is able to deposit the cell-carrying fluid onto different parts of the target area. In another example, the printing mechanism may remain static whilst the carrier and target area move in the x and/or y directions underneath the printing mechanism to facilitate the deposition of cell-carrying fluid onto different parts of the target area.

The cell deposition apparatus may further comprise a lift mechanism configured to adjust a distance between the transportation system and the imaging system. In particular, the lift mechanism may be configured to adjust a distance between the target area and the imaging system, preferably adjusting the distance when the target area is in the imaging position. As well as detaching the target area from the transportation system, which may affect the stability of the target area during the imaging process, moving the target area towards the imaging system ensures that the target area is brought into focus before an image of the target area is taken.

It would be advantageous to be able to load and image multiple substrates, one after another, into the cell deposition apparatus so that multiple experiments can be performed on each substrate automatically one after another. The apparatus may therefore comprise an incubator which is preferably configured to store at least one substrate. Storing multiple substrates reduces the need for user intervention between a change of experiments as a first substrate can be removed from the cell deposition apparatus and a second substrate can automatically loaded into the cell deposition apparatus ready for printing. Furthermore, the incubator provides an environment for storage of substrates which require incubation time during the experiment before the drops on the substrate can be imaged at the end of the experiment.

In order to move the substrate with the target area between the printing mechanism, the imaging system, and the incubator, the transportation system may be arranged to move the target area between the printing position and/or the imaging position and an incubating position, in which the target area is located substantially within the incubator. The incubator may also be sealed and detachable for long-term incubation and replaceable with unoccupied or partially occupied incubators to resume workflow. In another embodiment, the incubator may be positioned in between the print system and the imaging system to facilitate repeated periods of printing and incubation where imaging is the endpoint.

The incubator may be sealed and detachable, and optionally replaced with an incubator unoccupied or partially occupied by substrates. The incubator may be positioned substantially between the print system and the imaging system,

The at least one light source may be capable of performing dark field microscopy or infrared spectroscopy. The at least one light source may be, but is not limited to, brightfield-, fluorescence-, infrared-, x-ray-, UV- and Raman-sources. The imaging system may comprise a plurality of light sources.

The apparatus as previously described does not preclude the use of a plurality of printing systems, plurality of imaging systems, plurality of incubators and plurality of transportation systems that are interconnected to allow for increased speed of data acquisition and throughput of samples, more complex workflows and the continued use of alternative printheads systems, imaging systems, incubators and transportation systems whilst other systems are in operation. Thus, in some examples, the apparatus comprises a plurality of printing systems, plurality of imaging systems, plurality of incubators and plurality of transportation systems which may be interconnected.

The apparatus is typically contained within a housing such that the housing surrounds the individual components of the cell deposition apparatus, including the transportation system, the printing mechanism, and the imaging system. Providing a housing helps maintain a constant environment within the housing, around the individual components of the cell deposition apparatus. Advantageously, the rate at which the cell-carrying fluid evaporates, as well as the rate at which the deposited cell-carrying fluid deforms, can be limited. Experimental integrity can therefore be maintained, as well as allowing the user to set and control the internal environmental conditions of the housing, depending on the experiment being undertaken.

The apparatus may comprise a control system arranged to control at least one environmental parameter within the housing, such as temperature, pressure, humidity. The control system may be a computer-controlled system which can be initially programmed by a user before the experiment begins. As well as controlling the internal environment of the housing, the control system may be arranged to control the individual components of the cell deposition apparatus, including the transportation system, the printing mechanism, and the imaging system. Thus, all the individual components of the cell deposition apparatus may be computer controlled. The individual components may be controlled by a computer program which runs on the control system, the computer program being initially programmed by a user. This allows the user to initially set up the experiment and once the program has begun to run, no further interaction from the user is required. Thus a fully automated, computer controlled system can be provided.

The user may interact with the control system via a user interface, which may form part of the control system. The user interface may therefore be configured to allow a user to interact with at least one component of the apparatus so that the at least one component can be programmed by the user in accordance with the experiment to be conducted.

The substrate is typically a rigid substrate. Typically the substrate is a discrete object, although in some cases the substrate has the form of a continuous track such as multiple discrete objects in tandem, a belt or section from a roll of material. The substrate may be transparent to allow light transmission. The substrate may be transparent to visible light to allow imaging of the cell-carrying fluid on the substrate using bright field microscopy. The substrate may be opaque or reflective for use with other forms of illumination.

The imaging system may comprise a scanner, which is typically a digital scanner. Preferably the digital scanner is formed from a digital microscope which may be configured to carry out high resolution bright field microscopy.

The imaging system may comprise at least one light source. Preferably, the at least one light source may be disposed substantially opposite the scanner of the imaging system. In this case, by opposite we mean that the scanner has a line of sight which has a longitudinal axis and the light source is located along this longitudinal axis. Examples of other primary or additional secondary light sources include but are not limited to fluorescence, infrared, x-rays, UV and Raman sources.

The components of the apparatus which become exposed to the cell-carrying fluid may be sterilisable or disposable (for example biodegradable or recyclable), which may allow the apparatus to be used for multiple different sequential experiments.

In accordance with a second aspect of the invention there is provided a method of depositing and imaging a cell on a substrate, the method comprising: receiving, via a printing mechanism comprising at least one channel, a sample of cell-carrying fluid comprising at least one cell; depositing, via the at least one channel of the printing mechanism, the sample of cell-carrying fluid onto a target area of a substrate; moving the target area between a printing position, in which the target area is located substantially opposite the printing mechanism, and an imaging position, in which the target area is located substantially opposite an imaging system; and imaging all of the target area substantially instantaneously by moving the target area relative to an imager, wherein the imager is part of the imaging system and the imager is capable of imaging a region of a substrate wherein the region is smaller than the target area and the imaging system.

Preferably the region of the substrate being imaged by the imager is substantially smaller than the target area, and further preferably, the imaging system uses at least one light source compatible with imaging the samples deposited in the target area of the substrate.

The sample of cell-carrying fluid may comprise at least one cell, biomolecule or non-biological microparticle.

The depositing may comprise depositing, via the at least one channel of the printing mechanism, the sample of cell-carrying fluid onto a target area of a substrate in discrete or co-located coordinates.

In some alternative examples the imaging all of the target area substantially instantaneously comprises moving imager relative to the target area.

Preferably, the imaging time is commensurate with the time to deposit samples over the same region.

In some examples the method may be a method of depositing and imaging a biomolecule on a substrate, comprising the previously described method steps Preferably the above described method is configured to be carried out using the apparatus of the first aspect of the invention.

In some examples, there may also be an additional pre-deposition step wherein the receiving channels for the printheads may form part of a cell-sorting device such as but not limited to a FACS, MACS or a flow cytometer, which may or may not be integrated into the apparatus.

There may also be provided a computer program comprising instructions which, when executed by a computer, cause the computer to carry out the above described method.

There may also be provided a computer readable storage medium comprising instructions which, when executed by a computer, causes the computer to carry out the above described method.

The cell deposition apparatus therefore provides a complete “lab-in-a-box” system that is able to prepare complex biological experiments by depositing and layering thousands of variable drops in the femtolitre-to-microlitre range from multiple liquid input sources using the high-precision, repeatability, and high-throughput of the digital printheads.

The apparatus provides the ability to simultaneously test hundreds of cell, biomolecule or non-biological cell-mimicking microparticle variables in the femtolitre-to-microlitre drop-volume range, sequentially layered over each other by high-precision printheads, in multiplicate using typically only one assessment medium (for example a substrate), giving the ability to complete an entire experimental program in one process. Advantageously, more than one parameter can be altered in one set-up, with dose-curve/gradient responses simply integrated by variable repeated drop-on-drop printing. The result is that thousands of statistically relevant repeats of multiple facets of an experiment can be completed simultaneously in one run. There is a further benefit in that an unstained or unlabelled biological experiment may be set up as previously described above and allowed to progress to the desired point before ink-jetting or washing-in of biomarkers or stains, which may or may not arrest biological processes, and can be accurately applied by overprinting immediately and substantially instantaneously prior to imaging.

Optical assessment by digital swathe-scanning gives a ‘snapshot’ that measures multiple drops at the same time, thus eliminating time as a significant variable when compared to analysis of individual drops. Furthermore, digital imagery as an output feeds neatly into automated software solutions, which can be designed to complement the core technology and provide real-time analysis and trending, providing insight to experimental optimisation or new investigative routes. Digital imagery is also a convenient medium for data archiving, providing a format than can be easily transferred to collaborators, be fed into software analysis (both in situ and retrospectively), and be used for novel publication and presentation purposes.

By combining complex, variably layered, high-throughput bioprinted drops of femtolitre-to-microlitre volume with digital swathe-scanning the workflow efficiency is increased. This is because the combination of these advanced techniques into one system eliminates workflow compromises as, after preparation of starting materials, a (semi-) skilled technician can simply input the design of the experiment into the device's software and walk-away, leaving the machine to complete the required activities as a black-box process. Thus, key laboratory tasks are combined in a repeatable format that allows for black-box use and increased walk-away time, whilst maintaining subject viability and eliminating analysis time variation as a caveat to results.

The above described apparatus therefore provides a complete high-throughput product that ‘micro-arrays’ by biological printing pico-to-microlitre drops of solutions (similar to digital inkjet printing), layers the drops for increased experimental complexity and efficiency, and performs high-resolution optical imaging for direct analysis of tissues, cells, biomolecules, and non-biological microparticles. The technology proposed here will facilitate the automation of all these common lab processes to improve the throughput, accuracy and workflow of whole-lab activities and reduce the human labour and time required to complete current equivalent experimental steps.

### BRIEF DESCRIPTION OF DRAWINGS

Preferred features of the present invention will now be described by way of example only with reference to the accompanying drawings in which:

FIG. 1a shows a schematic cross-section view of a first example of a cell deposition apparatus;

FIG. 1b shows a schematic cross-section view of a first example of a cell deposition apparatus including an attached bioreactor and user interface;

FIG. 2 shows a schematic cross-section view of a second example of a cell deposition apparatus including an incubator;

FIG. 3a shows a schematic cross-section view of a third example of a cell deposition apparatus; and

FIG. 3b shows a schematic cross-section view of fourth example of a cell deposition apparatus.

### SPECIFIC DESCRIPTION

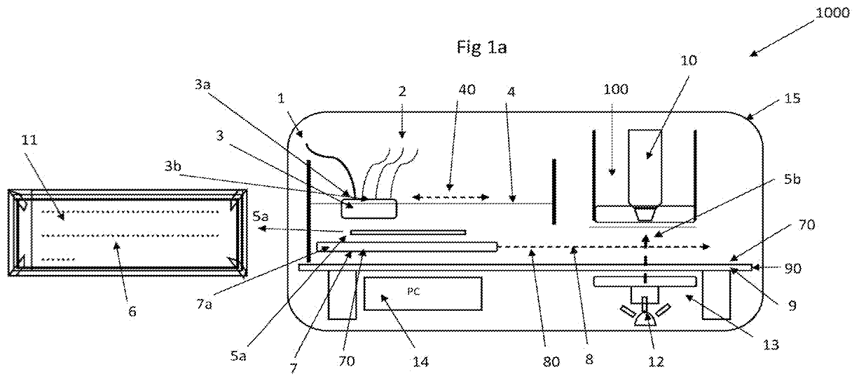

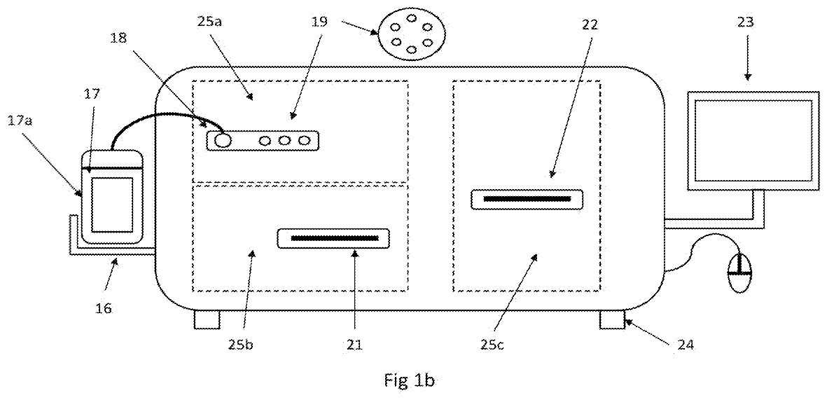

FIG. 1a shows an example of a cell deposition apparatus 1000. The apparatus 1000 comprises a housing 15 which surrounds a printing mechanism 3, for receiving and printing biological material in fluid form, a transportation system 70 for moving the printed biological material within the housing 15, and an imaging system 10 for imaging the printed biological material. In use, the printing mechanism 3 prints the biological material onto a target area 11, which may also be referred to as a sample area, of a substrate 5a which is imaged by the imaging system 100.

Biological fluids are fed into the printing mechanism 3 to be printed as drops 6 onto the substrate 5a. The biological fluid is made up of a cell input 1 and at least one liquid biochemical input 2. In some cases more than 2 liquid biochemical inputs are used. The input may also take the form of a non-biological fluid containing cell-mimicking microparticles for calibration purposes (not shown). The cell input 1 is located within its own carrier fluid, which is the liquid medium the cells are kept alive in. Each of the biochemical inputs 2 will be in their own carrier fluids i.e. solutions that stabilise the biochemical and may or may not be toxic to the cells in the cell input. In general, the aim of experiments will be to investigate effect of the biochemical inputs 2 on the cell-input 1. However input 2 can also contain cells, to facilitate experiments assessing the impact of one cell type on another.

The cell input 1 is held in solution, which acts as the printing ink, so that the cells can be printed. The solution used must therefore be compatible for printing and also provide a suitable environment for suspended the cells of the cell input 1. This imposes certain restrictions on the properties of the solution, for example the viscosity of the solution, so that the cells can be printed on the substrate 5a. Suspending the cells of the cell input 1 in solution prevents the cells from clumping together, ensuring that individual cells can be scanned by the imaging system 100 if required.

The choice of biochemical input 2 may depend on the particular choice of cell used for the cell input 1. For example, if mammalian cells are used as the cell input 1, the biochemical input 2 must be in a suitable carrier fluid for carrying mammalian cells, without damaging the cells. For example, nucleic acids and amino acids used as inputs in mammalian growth media as carrier fluids for mammalian cells. The specific choice of biochemical input 2 should replicate the natural environment and conditions in which the particular cells of the cell input 1 would typically be found. For example, if liver cells are used as the cell input 1, then the liquid biochemical input 2 should replicate liver growth conditions. If, instead, blood cells are used, the liquid biochemical input 2 should replicate blood conditions. It is important to replicate the natural conditions of the input cells to ensure that the identity of the input cell 1 does not change as a result of the biochemical carrier fluid used, but remains the same throughout the printing and scanning process, unless cell fate or differentiation is the parameter under investigation.

If the biochemical input 2 is not selected for the particular choice of input cell, the identity or viability of the input cell may be changed. That is to say, if a liver cell is carried in blood-like fluid, the liver cell may not survive in these conditions, or differentiate, and stop being the same as the liver cell that was input. This will clearly have detrimental effects on the biology experiment if the input cells do not remain constant throughout, unless this is the aim of the experiment.

The printing mechanism 3 comprises a printhead unit 3a, which is suitable for receiving and printing biological fluids and may be referred to as a biocompatible printhead unit. The printhead unit 3a comprises an array of individual printheads 3b each individual printhead 3b having a single printing channel. The printhead unit 3a can therefore be thought of as a multi-channel printhead unit. The individual printheads 3b of the printhead unit 3a are arranged in a 2-dimensional n×m array, where n and m represent numbers of individual printheads 3a for example a 2×2 array. However, other array configurations could also be used, for example a 1-dimensional array of n individual printheads 3b. The multi-channel printhead unit provides the ability to deposit multiple drops from multiple inputs without the need for manual intervention. In other examples, the printhead unit 3a can instead comprise a multi-channel printhead (not shown), i.e. a single individual printhead having multiple printing channels.

Each printing channel is used to carry a corresponding fluid through the individual printheads 3b and selectively print the corresponding fluid onto the substrate 5a. The channels may form part of a cell-sorting device such as but not limited to a FACS, MACS or flow cytometer that is upstream of the printhead unit 3a, which may or may not be integrated into the apparatus (not shown).

The solution including the cell input 1 and the liquid biochemical input 2 are therefore fed into separate printing channels within the printhead unit 3a and the fluids are not able to mix inside the printhead unit 3a. Instead, the two input fluids 1, 2 are mixed on the substrate 5a once each fluid has been selectively printed. The printhead unit 3a therefore has the ability to intentionally connect, or wick, drops of fluid together by printing subsequent drops of fluid on top of or next to a previously printed drop. This allows the amount of each fluid 1, 2 to be selected prior to printing so that the relative quantities of each fluid to be printed on the substrate 5a can be chosen depending on the user's experiment.

However, in some examples, the fluids can be mixed within the printhead unit 3a and printed on the substrate 5a as a mixture. In some examples the cell-input 1 or the biochemical input 2 can be replaced by a fluid containing non-biological microparticles that mimic the cell-input 1 or the biochemical input 2, such as for the purposes of manufacturing and calibration processes.

The printhead unit 3a is mounted on a carrier mechanism 40 in the form of a printing track 4. The carrier mechanism 40 allows the printhead unit 3a to be moved relative to the position of the substrate 5a. As shown in FIG. 1a, the printhead unit 3a is configured to move laterally within a horizontal plane within the housing 15. The printhead unit 3a is able to move back-and-forth in the x-direction, as well as to-and-fro in the y-direction. This allows the printhead unit 3a to be positioned over different parts of the substrate 5a, which is located generally below the printhead unit 3a but not necessarily directly below the printhead unit 3a, so that the printhead unit 3a is able to print onto different target areas 11 on the substrate 5a. Although the printhead unit has been described as moving relative to a static substrate, in some designs, the substrate will be moved relative to a static printhead unit in both the x- and and y-directions.

Being able to move the printhead unit 3a relative to a printable surface area of the substrate 5a means that the size and the location of the target area 11 on the substrate 5a can be chosen by the user. In some cases the target area 11 will represent a small portion of the total printable surface area of the substrate 5a and in other cases the target area 11 will represent a majority of the total printable surface area of the substrate 5a. In general, the target area 11 is large enough so that it can receive thousands of individual drops 6, each drop 6 being in the nano- to pico-litre range, without unintentional wicking or wetting together of the individual drops 6 but small enough that the entire target area 11 can be quickly scanned using minimal number of scans, as will be explained in more detail later.

All the individual printheads 3b which make up the printhead unit 3a are fixed relative to each so that the entire printhead unit 3a moves as a single unit. All the individual printheads 3b of the printhead unit 3a are therefore mounted on one single printing track 4. This ensures that all the individual printheads 3a are moving together and are in the same location at the same time, relative to the target area 11 on the substrate 5a. This also reduces the number of mechanisms required to move during operation of the apparatus. In some embodiments, the individual printheads 3b within the printhead unit 3a are able to move relative to each other. This allows the spacing between individual printheads 3b, and consequently the spacing between each deposited drop, to vary on the target area 11. This may be advantageous when the drops deposited from each printhead 3b are not equal in size and so the spacing between different drops needs to be varied on target 11, depending on the size on the drop.

Mounting the printhead 3 on a printing track 4 allows each fluid 1, 2 to be printed on top of each other at the same location within the target area 11 on the substrate 5a, in order that the fluids can be mixed on the substrate 5a. The printing track 4 therefore allows multiple fluid layers to be printed onto the substrate 5a.

A particular print location on the substrate 5a can be identified using xy-coordinates. Each printed drop 6 on the substrate 5a is therefore associated with a set of xy-coordinates. These coordinates can then be used to make the printhead unit 3a print drops 6 of the fluids 1, 2 in the same location as a previously printed location on the substrate 5a or in a different location.

The size of the drop 6 printed by the printhead unit 3a depends on the size of the cell used as the cell input 1. For example, larger cells require larger drop sizes compared to smaller cells. Typically, drops of the order picolitres or nanolitres are used.

The substrate 5a onto which the fluid drops are printed is a rigid substrate which is compatible with the biological experiment under consideration. The substrate 5a is sized to receive many multiples of nanodrops 6 from alternating fluid inputs 1, 2 when positioned under the array of printheads 3. Once the substrate 5a has been printing with fluid drops 6, it may be referred to as a bio-printed substrate.

The substrate 5a is a discrete object in the form of a microscope slide, for example based upon a borosilicate glass slide, as these are readily available and suitable for most general applications. However, as will be appreciated, the substrate composition can be selected in order to be compatible with individual experiments, for example the substrate could be glass or plastic, flat or indented with microwells and structures. Furthermore, cell input 1 or biochemical input 2 could be replaced by a cell-containing or non-cell-containing liquid capable of gelling or solidifying upon deposition to allow customisation of the surface of the substrate with biocompatible structures (not shown).

It is potentially advantageous to treat the printable surface of the slide which will receive the printed fluid to prevent the drops 6 from spreading out over the surface of the substrate 5a after it has been printed. One example of treatment might be siliconization, such as with dichlorodimethylsilane, to make the surface of glass hydrophobic and therefore prevent aqueous droplet spreading whilst enabling light transmission. However, it will be appreciated that different treatment methods and chemicals can be used depending on the compatibility with the substrate, droplet contents and illumination method. For example, if fluid is printed straight onto a glass substrate, the fluid drop may not remain sufficiently localised as a single discrete drop but is likely to spread out over the surface of the glass and merge with other drops that have already been printed on the substrate 5a. Treating the substrate 5a first ensures that the drops remain as discrete drops, such as by siliconisation treatment hydrophobically repelling liquid. Treating the printable surface of the substrate 5a therefore controls the spreading of the drops 6 after deposition through surface energy. As already explained, mixing can be achieved, if desired, by printing multiple drops of the same or different fluid on the same printing location using the xy-coordinates.

As explained, once the biological material has been printed, the target area 11 of the substrate 5a is moved from the printing location to the imaging location using the transportation system 70. The transportation system 70 includes a support mechanism 7 for supporting the substrate 5a, a motion system 80 for moving the support mechanism 7 between the printing and the imaging positions, and a main frame 90 having a frame base 9, to which the support mechanism 7 and motion system 80 are attached. In some developments, there is a further optional stage prior to movement by transportation system 70 in that an unstained or unlabelled biological experiment may be set up as previously described above and allowed to progress to the desired point before ink-jetting or washing-in of biomarkers or stains by printheads 3b, which may arrest biological activity, can be applied by overprinting immediately prior to movement of substrate 5a to imaging system 100 by transportation system 70.

The frame base 9 extends substantially across the internal area of the housing 15 underneath each of the printhead unit 3a and the imaging system 100 so that the support mechanism 7 can be moved between the printing position, in which the support mechanism 7 is located below the printhead unit 3a, and the scanning position, in which the support mechanism 7 is located below the imaging system 100.

The main frame 90 of the transportation system 70 rests on a floor of the housing via a plurality of supporting feet. As the frame base 9 is raised off the floor of the housing 15 a cavity space is created underneath the frame base 9. This space may be used to house key components such as a computerised control system 14 and motors which power the apparatus 1000.

The computer control system 14 is connected to all the individual components of the cell deposition apparatus 1000 including the printing mechanism 3, the transportation system 70, the imaging system 100, and all the sub-components of these systems. All the individual components and sub-components of the apparatus 1000 are therefore computer controlled, providing a full automated, computer controlled apparatus. A computer program runs on the computer control system, which can be programmed by a user. The user is able to input the initial conditions and details of the experiment into the computer program so that when the program is run, the cell deposition apparatus carries out the required experiment without any further interaction from the user, until the experiment has been completed.

As shown in FIG. 1a, the support mechanism 7 is in the form of a receiving platform 7a and the motion system 80 is in the form of a shuttling mechanism 8. The substrate 5a is therefore positioned on, and supported by, the receiving platform 7a. The receiving platform 7a is attached to the shuttling mechanism 8, which moves the receiving platform 7a within the housing 15. The shuttling mechanism 8 moves the receiving platform 7a laterally within the housing 15 of the apparatus 1000, the movement being confined to a single horizontal plane. The receiving platform 7a can therefore move left and right, between the sides of the housing 15, as well as forwards and backwards, between the front and rear of the housing 15. The shuttling mechanism 8 is therefore a multi-directional shuttling mechanism, for example an X+Y shuttling mechanism, which spans the frame base 9 of the main frame 90.

The bio-printed substrate, supported by the receiving platform 7a, is shuttled back-and-forth between the printhead unit 3a and the imaging system 100. The shuttling mechanism 8 ensures that the substrate 5a is accurately positioned underneath the imaging system 100, the shuttling mechanism 8 allowing the position to be finely tuned if necessary in the x- and y-directions.

The imaging system 100 includes a scanner, which is a digital scanner 10 in the form of a digital microscope. The printed drops 6 of biological fluid on the substrate 5a are imaged using bright field microscopy, in which a light source 12 is positioned beneath the digital scanner 10 and arranged to shine light towards the digital scanner 10 along a vertical light path 5b. The light illuminates the biological sample on the substrate from behind, giving a transmission image on a bright background (as seen through the digital scanner 10). In order for the structure of the cells of the biological material to be seen clearly, some cells are potentially pre-stained. Staining the cells allows the use of white spectrum light, which is commonly available as a light source. Bright field microscopy therefore provides a high-resolution image (capable of at least ×40 optical magnification) which allows individual cells, including some bacteria, to be resolved.

In some cases, dark field microscopy is used instead which does not require the cells to be stained. This technique is a lower-resolution imaging technique than bright field microscopy, which is high-resolution, and so detailed images of the internal structure of the cells aren't captured. However, dark field microscopy is useful for experiments in which high-resolution is less relevant to the results required, such as for quickly counting the number of cells present and identifying cell boundaries, i.e. for experiments where high levels of details of the cell structure is less important. This may also be achieved through the integration and application of IR spectroscopy. In some cases, other light sources can be used to detect other methods for labelling and staining cells, such as but not limited to fluorescence, infrared, x-ray, UV and Raman sources.

For successful bright field microscopy, it is therefore important that both the substrate 5a and the receiving platform 7a are transparent to light so that neither the substrate 5a nor the receiving platform 7a obstructs the light path 5b between a light source 12 and the digital scanner 10 when the receiving platform 7a is in the scanning position.

The receiving platform 7a and substrate 5a are therefore constructed from materials which transmit visible light. Alternatively, the receiving platform 7a could include an aperture (not shown) which allows the light path 5b to pass through the receiving platform 7a and subsequently through the transparent substrate 5a. Thus, when the receiving platform 7a is in the scanning position, the aperture in the receiving platform 7a is directly above the light source 12 so that the receiving platform 7a does not obstruct the light path 5b, but instead the light path 5b transmits through the aperture.

In some examples, the surfaces of both the receiving platform 7a and the substrate 5a could instead be reflective and the light source 12 could be positioned above both the receiving platform 7a and the substrate 5a, next to the digital scanner 10, to illuminate the biologically printed drops 6 from above.

The number of drops 6 printed on the substrate 5a can be varied, depending on the experiment being undertaken. For example, the same experiment may be carried out on the same drops multiple times, the same drug might be used on different printed drops, or different concentrations of drugs might be used for different drops. The relative drop size might also be important as the size of the drop can be used to change to concentration of the drug being used in a particular experiment. In general, a larger drop size will correspond to a lower concentration of drug, for a given mass of drug per drop, as the drop is more diluted.

To ensure that the substrate 5a is horizontal during the scan, a lift mechanism 13 engages with the receiving platform 7a and the substrate 5a, and brings the substrate 5a into a horizontal position. This is achieved by having a plurality of upstanding prongs on the receiving platform 7a onto which the substrate 5a is initially placed, before printing. When the receiving platform 7a is in the scanning position, the lift mechanism 13 lifts the substrate 5a vertically off the prongs and adjusts the position of the substrate 5a, if necessary, so that the substrate 5a is horizontal. The angle of the substrate with respect to the horizontal is adjusted by changing the pitch and tilt of the plane of the substrate until the plane of the substrates coincides with the horizontal plane.

As well as detaching the target area 11 on the substrate 5a from the receiving platform 7a, which may affect the stability of the target area 11 during the imaging process, the lift mechanism moves the target area towards the digital scanner 10 so that the target area is brought into focus before the digital scanner 10 images the target area 11. Alternatively, the digital scanner 10 can be mechanically manoeuvred vertically into position above the substrate 5a. In either embodiment, fine focus will be achieved through the focal mechanisms of the digital scanner 10.

The digital scanner 10 then scans the horizontal substrate 5a. After scanning has been completed, the lift mechanism 13 lowers the substrate 5a back onto the prongs on the receiving platform 7a.

As the lift mechanism 13 is located between the light source 12 and the digital scanner 10, it is important that the lift mechanism 13 is constructed so that it does not obstruct the light path 5b between the light source 12 and the digital scanner 10. The light source 12 is therefore still able to illuminate the back of the substrate 5a holding the drops 6 without the lift mechanism 13 interfering.

As mentioned, once the drops 6 of biological fluid have been printed onto the substrate 5a, the receiving platform 7a is moved, via the shuttling mechanism 8, from underneath the printhead unit 3a to underneath the digital scanner 10. The light source 12 positioned underneath the digital scanner 10 and the receiving platform 7a provides the illumination required for bright field scanning.

The digital scanner 10 is arranged to perform swathe-scanning across the entire sample area 11 of the substrate 5a. Swathe-scanning involves scanning multiple drops 6 of fluid 1, 2 at the same time when the sample area 11 is larger than the field of view (FOV) of the digital scanner 10.

The proportion of the total surface area of the substrate 5a which can be viewed by the digital scanner 10 at one time is determined by the FOV of the digital scanner 10. Thus, the digital scanner 10 is only capable of imaging a region of the substrate 5a when the substrate 5a is in a static position, this region being, in general, less than the total surface area of the substrate 5a. The FOV of the digital scanner 10 therefore determines what percentage of the surface area of the substrate 5a can be imaged at one time when the substrate 5a is stationary.

In general, the target area 11 onto which drops 6 are deposited will be larger than the FOV of the digital scanner 10. This means that the digital scanner 10 is only able to view a limited proportion of the total number of drops 6 in the target area 11 at a time. In order to image all the drops 6, the FOV of the digital scanner 10 needs to be moved over the entire sample area 11 so that all the drops 6 can be imaged.

During the swathe-scan, the entire target area 11 moves very quickly under the digital scanner 10. The total number of drops 6 scanned per swathe is given by the number of drops per field of view multiplied by the number of individual field of views. For example, a target area 11 contains 2 columns of drops, each column having 100 rows, and the FOV of the scanner 10 can view 2 drops at a time (i.e. one complete row of drops). The swathe-scan will move substantially instantaneously across all 100 rows, imaging each pair of drops in each row, so that 1 swathe-scan represents 2×100=200 drops captured in one single image-swathe. In reality, there will be a negligible time difference between the time when the first row of 2 drops was scanned and the time at which the last (i.e. 100th) row of 2 drops was scanned.

The digital scanner 10 detects what proportion of the substrate 5a the target area 11 covers so than when the swathe-scan is performed, the entire target area 11 is captured. The digital scanner 10 is therefore able to detect when cells have been printed in different locations on the substrate 5a and ensures that all the deposited cells are scanned.

The scan time is commensurate with the relative time to deposit for the same area within a reasonable system timeframe. In some examples, the swathe scan can be captured within a few microseconds. This has the effect that the time at which an initial part of the target area 11 is scanned is substantially the same as the time as which a final part of the target area 11 is scanned. This ensures that there is no substantial time difference during the length of the scan compared to its deposition so that an entire experiment, represented by the total target area 11, can be scanned substantially instantaneously. This allows the effects of different drugs on different cells to be analysed more effectively because the length of time over which the drug acts on each cell is now substantially a constant instead of a variable.

The negligible time difference is important because it means that there is less variance as a result of the act of capturing the data in the first place. If, for example, a user was to manually perform the same experiment it would take them a long time to keep adjusting the position of the target area 11 on the substrate 5a to ensure that all the drops 6 were imaged. The imaging would therefore need to be performed multiple times over the entire target area 11, which takes time, increasing the likelihood of collecting different results as a result of the drug acting for a longer time on some cells than others. The user would then have to filter through these results and discard the ones for which the time difference is too significant or accept a degree of inaccuracy.

The swathe-scan is a continuous, rapid movement. Provided all the parts of the equipment are stabilised, there are no visualisation issues i.e. the captured scanned image is not blurry as a result of the rapid movement of the scanner 10. As will be appreciated, different swathe-scans can be combined together using algorithms which identify the edges of different swathe-scans and match up the edges of consecutive swathe-scans to produce a final, large, overall image of the entire experiment undertaken.

The digital scanner is therefore able to collect high-resolution imagery of the whole target area 11 of the substrate in swathes. This swathe-scanning technique is intended for the analysis of printed cells or pre-seeded cellular treatment analysis.

The digital scanner 10 can also be equipped with fluorescent microscopy capabilities for biomolecular studies, such as protein-protein interactions or the detection of specific gene and protein expression from cells. A fluorescent light source is advantageous for multi-wavelength signal detection of marker biomolecules. In some cases, other light sources can be used to detect other methods for labelling and staining cells, such as but not limited to infrared, x-ray, UV and Raman sources.

As mentioned, the housing 15 encases all the individual components of the apparatus 1000 including the transportation system 70, the printing mechanism 3, and the imaging system 100. The housing 15 maintains a constant environment surrounding the individual components, which means that, in particular, the rate of evaporation of the biological fluid as well as the rate of drop deformation can be limited. This helps maintain experimental integrity and allows the user to initially set and control the internal environmental conditions of the housing 15, depending on the particular experiment being conducted. The housing 15 rests on raised feet 24, as can been seen in FIG. 1b which allows for ventilation and heat dissipation from the housing 15 into the surrounding environment, helping maintain the internal temperature within the housing 15.

As the apparatus 1000 is sealed from the external environment by the housing 15, the cells in the cell input 1 must be carefully transferred from outside the housing 15 to the printing mechanism 3 inside the housing 15 without disrupting the internal housing conditions.

To overcome this potential problem, the cell input 1 is taken from a cell storage compartment 17, which may be in the form of a bioreactor 17a or a cell-sorter such as a FACS, MACS or flow cytometer (not shown), which is attached to the side of the housing 15, as shown in FIG. 1b. A bioreactor 17a would also perform the role of maintaining clump-free and equal distribution of cells in a cell-carrying fluid and mitigate blockages in input 18 or printheads 3b. The storage compartment 17 is attached using any suitable attachment means 16, for example a bracket or frame. The storage compartment 17 may also be free-standing in other examples. The storage compartment 17 is connected to a feed system (not shown) which allows the storage compartment to feed directly into an input 18 of the printing mechanism 3 and into the channels of the individual printheads 3b in the printhead unit 3a, ready to be deposited, or printed, onto the target area 11 on the substrate 5a. The feed system includes a plurality of tubes, which connect the storage compartment 17 to the printing mechanism 3, and at least one pump which transfers the cell in the cell input 1 from the storage compartment 17, through the tubes, and into the printing mechanism 3.

In some examples, instead of directly feeding the cell input 1 from the storage compartment 17 into the printhead unit 3a, low-volume biochemical inputs can be taken directly from external syringe ports 19, or a multi-port wheel 20, and fed into the printhead input 18 via a series of tubes (not shown).

At least one control hatch provides sterile access to the substrate loading mechanism 21 as well as providing direct access to the digital scanner control panel 22 to allow the user to control the function of the digital scanner 10. A substrate loading mechanism 21 is generally a slot or opening that allows a substrate to be either pushed or pulled into position in the apparatus interior. That is, a substrate loading mechanism 21 is any suitable process that can be used to get a substrate from outside to inside the apparatus. An advantage of providing at least one control hatch is that a user is able to rapidly access each component and its corresponding control panel individually. In some cases, each control panel is associated with a distinct, separate control hatch but in other cases one control hatch may be used to access several control panels at the same time.

A computer monitor 23 is connected to the control panels and components of the cell deposition apparatus 1000, allowing the user to interact with and control the various different components via a user interface. The user interface may be in the form of a touchscreen, a screen-and-mouse attachment, or any other suitable interaction mechanism, and forms part of the control system.

As well as user accessible control hatches, a number of service hatches 25a-c are also provided in the housing 15. These provide service engineers with quick and easy access to the core components of the apparatus.

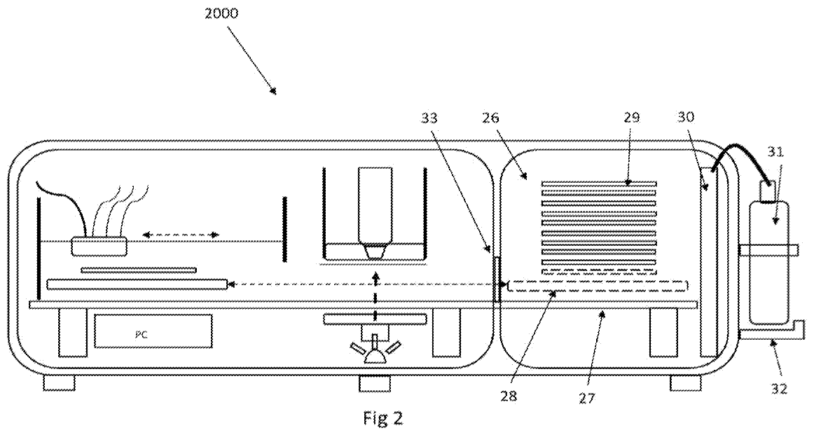

In another example apparatus 2000, a biological incubator 26 is included within the housing 15, as shown in FIG. 2. The incubator 26 forms an extension of the apparatus components and is located next to the imaging system 100. This allows the receiving platform 7a to deliver the bio-printed substrate, supported by the receiving platform 7a, from either the printing mechanism 3 or the imaging system 100 to the incubator interior 26.

As is shown in FIG. 2, the incubator 26 is an extension of the transportation system 70 with the frame base 9 of the transportation system 70 extending into the cavity of the incubator 26. The printing track 4 on which the receiving platform 7a moves also extends into the cavity of the incubator, above the frame base 9, so that there is one continuous printing track 4 which is able to serve the printing mechanism 3, the imaging system 100, and the incubator 26.

In an alternative example, there could be two separate printing tracks, a first track which serves the printing mechanism 3 and the imaging system 70 and a second track which serves the incubator 26. In this case, the receiving platform 7a would need to be transferred from the first track to the second track, via a transfer system, when the receiving platform 7a is to be moved into the incubator 26. Whilst this arrangement may require more individual components, it may allow for a modular cell deposition apparatus, allowing the incubator 26 to be attached and detached from the main body of the cell deposition apparatus as and when required.

The incubator 26 includes an automated stacking system 29 which allows multiple substrates 5a to be stacked within the cavity of the incubator 26. Although the multiple substrates 5a have been shown as vertically stacked in FIG. 2, it will be appreciated that the substrates 5a can be organised in any other convenient arrangement. Storing multiple substrates 5a in the incubator 26 allows the possibility of loading and scanning many different substrates one after another, allowing multiple experiments to be performed automatically one after another, without the need for user intervention between changing subsequent substrates 5a.

The incubator 26 maintains the temperature, humidity and hypoxic environment of the internal cavity of the incubator 26 where the substrates 5a are stored. An external gas cylinder 31, for example a carbon dioxide cylinder, can be fluidly connected 30, for example via at least one pipe or tube, to the internal cavity of the incubator 26 for gas regulation. Any suitable attachment means 32, for example a bracket or frame, is attached to the outside of the housing 15 to support the gas cylinder 31. Other environmental factors inside the incubator 26 can also be controlled including, for example, vapour pressure, dust reduction, and atmospheric pressure.

In order to maintain the controlled environment inside the incubator 26 whilst still allowing the platform 7 to enter and leave the incubator cavity, a fluid-tight hatch 33 is provided between the main compartment, which includes the printing assembly 3 and imaging systems 100, and the incubator as shown in FIG. 2. When the platform 7 is ready to be transferred from the main compartment into the incubator 26, this hatch 33 is briefly opened to allow the platform with its corresponding substrate 5a to enter the incubator 26.

This arrangement allows for more extensive, automated experiments for example allowing for cells to adhere to the substrate followed by automated analysis or when iterative seeding of treatment of the cells is required with time-course analysis. This arrangement therefore increases the complexity of possible experiments which can be performed and facilitates workflow. Once the apparatus has been initially programmed by the user using the computerised control system 14, the apparatus can be left to automatically process multiple experiments with little-to-no further human interaction for long periods of time.

In a further arrangement of the apparatus (not shown), the incubator 26 may also be sealed and detachable for long-term external incubation and replaceable with unoccupied or partially occupied incubators 26 to resume or change workflow. In another arrangement, the incubator 26 may be positioned in between the printhead array 3 and the imaging system 34 to facilitate repeated periods of printing and incubation where imaging is the endpoint.

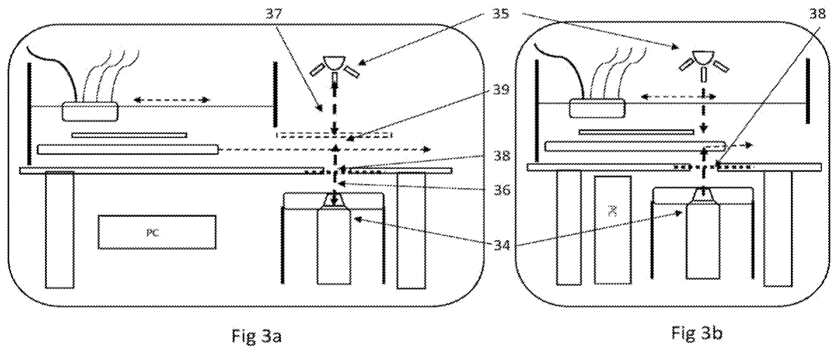

An alternative arrangement of a cell deposition apparatus 2000 is shown in FIG. 3a, where the same reference numbers represent components with the same function as has been previously described. As before, this alternative arrangement includes an imaging system 34 and at least one light source 35 but these are inverted with respect to the arrangement illustrated in FIGS. 1 and 2. Thus, in this case, the imaging system 34 is located below the frame base 9 and the at least one light source 35 is located above the frame base 9. The imaging system 34 is moveable along a vertical path 36 so that it can be extended upwards towards to the light source 35, when in use, and retracted downwards into the cavity underneath the frame base 9, when not in use. The light source 35 is also moveable along a vertical path 37 so that it can be moved downwards toward the frame base 9, when required during scanning, and moved upwards away from the frame base 9, when the scanning has been completed and the light source 35 is no longer needed.

An aperture 38 in the frame base 9 provides a clear, unobstructed path between the imaging system 34 and the light source 35 so that the substrate 5a can be analysed.

By rotating the configuration of the light source 35 and the imaging system 34 relative to each other, a portion of the focal plane is removed making it easier for the scanner to analyse the cells 1 on the substrate 5a because there is no depth of substrate 5a. Thus, an advantage of this construction is that there is a consistently flat focal point via the flat base 39 of the substrate 5a against which cells 1 would directly settle and adhere to, as opposed to a varying surface depth in the previous described arrangement, which increases the focal reliability. The design in FIG. 3a therefore enhances the optical path. Furthermore, this setup only has one focal plane and so the focal tracking mechanism has fewer calculations to do.

FIG. 3b shows another example in the arrangement of the apparatus where, again, the same reference numbers represent components with the same function as has been previously described. In this arrangement, the light source 35 and imaging system 34 are inverted relative to the arrangement in FIGS. 1 and 2 (i.e. the same as that shown in FIG. 3a), but in this case the overall footprint of the apparatus has been reduced. This is achieved by having the horizontal-moving printhead array 3 and platform 7 condensed into the same volume as the vertically-moving light source 35 and imaging system 34. In this arrangement the aperture 38 in the frame base 9, through which the optical path travels, overlaps with the printing track 4 along which the platform 7 travels.

In another arrangement of the apparatus (not shown) a plurality of printheads arrays 3, a plurality of imaging systems 34 and a plurality of incubators 26 can be connected by a plurality of transportation systems 70. This serves the purpose of increasing the throughput of the apparatus and facilitates increasing complexity of workflow and the continuous use of alternative printhead arrays 3, imaging systems 34 and incubators 26 whilst other printhead arrays 3, imaging systems 34 and incubators 26 are in operational use.

To use the apparatus, the user starts by selecting the type of experiment to be carried out using the user interface of the computer control system. The computer system will then select the initial start conditions for the experiment about to be undertaken, including the internal conditions in the incubator and the housing. In some cases, the user may additional select, or control, the initial experimental condition via the user interface.

Once the apparatus has been programmed, the user loads at least one substrate into the apparatus on which the experiment will be printed and conducted.

The user can then initiate the computer program to carry out the experiment and no further actions from the user are required, until the computer control system alerts the user that the experiment has been a completed, or a problem has been encountered such that the experiment cannot be completed.

To start the experiment, a substrate might be loaded from the incubator onto the receiving platform 7a inside the cell deposition apparatus. The printhead unit 3a of the printing mechanism 3 then receives a sample of cell input 1 and liquid biochemical input 2, the sample of cell input 1 including at least one cell. The individual printheads 3b of the printhead unit 3a then print the sample onto the target area of the substrate 5a in a series of individual drops 6.

Once the required number of drops 6 has been printed onto the target area 11, the substrate 5a is then moved, by the receiving platform 7a, from underneath the printhead unit 3a, across the frame base 9 of the main frame, to underneath the digital scanner 10.

The lift mechanism then engages with the substrate 5a, lifting it off the receiving platform 7a, and bringing it into focus with the digital scanner 10. The digital scanner 10 swathe scans the entire target area on the substrate 5a by moving very rapidly over the entire target area on the substrate 5a. Alternatively, substrate 5a is moved on platform 7a rapidly underneath a mechanically aligned but static digital scanner 10. The rapid movement has the effect that the scan is conducted almost instantaneously, despite the target area being larger than the field of view of the digital scanner 10 when there is no relative movement between the digital scanner 10 and the target area.

Once the scan has been completed, the substrate 5a is placed back onto the receiving platform 7a. The substrate can be then moved to the incubator for incubation and storage during long experiments.

The process then begins again, automatically, with the next substrate 5a until all the required number of substrates 5a have been printed. Thus, the constant cycling is automatically carried out and the user is not required to reload the apparatus each time or to manually move the substrate between different components of the apparatus in order for the experiment to be carried out.

1. A cell deposition and imaging apparatus comprising:
a printing mechanism comprising at least one channel, the at least one channel of the printing mechanism arranged to:
receive a sample of a cell-carrying fluid comprising at least one cell-type; and
deposit the sample of the cell-carrying fluid onto a target area of a substrate, an imaging system arranged to image the target area; and
a transportation system arranged to move the target area between a printing position, in which the target area is located substantially adjacent to the printing mechanism, and an imaging position, in which the target area is located substantially adjacent to the imaging system;
wherein the imaging system comprises an imager capable of imaging a region of the substrate wherein the region is smaller than the target area and the imaging system is arranged to image all of the target area by moving the target area relative to the imager.

2. The apparatus of claim 1 wherein the printing mechanism is arranged to receive multiple samples of cell-carrying fluid and deposit the multiple samples of the cell-carrying fluid onto a target area of a substrate.

3. The apparatus of claim 2 wherein the imaging system is arranged to image the multiple samples in the target area substantially simultaneously.

4. The apparatus of claim 1 wherein the printing mechanism comprises a plurality of printheads arranged as an array of printheads, each printhead comprising a channel.

5. The apparatus of claim 4 wherein the plurality of printheads are arranged to move together as a single unit.

6. The apparatus of either claim 1 wherein each channel in the array of printheads is arranged to receive a respective cell-carrying fluid to be deposited on a substrate, each cell-carrying fluid comprising at least one cell.

7. The apparatus of claim 1 wherein the printing mechanism is arranged to be mounted on a track to allow the printing mechanism to be moved along the track relative to the transportation system.

8. The apparatus of claim 1 further comprising a lift mechanism configured to adjust a distance between the target area and the imaging system when the target area is in the imaging position.

9. The apparatus of claim 1 further comprising an incubator configured to store at least one substrate.

10. The apparatus of claim 9 wherein the transportation system is arranged to move the target area between the printing position and/or the imaging position and an incubating position, in which the target area is located substantially within the incubator.

11. The apparatus of claim 9 wherein the incubator is positioned substantially between the print system and the imaging system.

12. The apparatus of claim 1 wherein the at least one light source is capable of performing dark field microscopy or infrared spectroscopy.

13. The apparatus of claim 1 wherein the imaging system comprises a plurality of light sources.

14. The apparatus of claim 1 wherein the apparatus is contained within a housing.

15. The apparatus of claim 14 further comprising a control system arranged to control at least one environmental parameter within the housing, for example temperature, pressure, humidity.

16. The apparatus of claim 1 further comprising a computer system arranged to control individual components of the apparatus, including the printing mechanism, the transportation system, and the imaging system.

17. The apparatus of claim 16 wherein the computer system further comprises a user interface configured to allow a user to interact with at least one component of the apparatus, including the printing mechanism, the transportation system, and the imaging system.

18. A method of depositing and imaging a cell on a substrate, the method comprising:
receiving, via a printing mechanism comprising at least one channel, a sample of cell-carrying fluid comprising at least one cell;
depositing, via the at least one channel of the printing mechanism, the sample of cell-carrying fluid onto a target area of a substrate;
moving the target area between a printing position, in which the target area is located substantially opposite the printing mechanism, and an imaging position, in which the target area is located substantially opposite an imaging system; and
imaging all of the target area substantially instantaneously by moving the target area relative to an imager, wherein the imager is part of the imaging system and the imager is capable of imaging a region of a substrate wherein the region is smaller than the target area and the imaging system.

19. A computer program comprising instructions which, when executed by a computer, cause the computer to carry out the method of claim 18.

20. A computer readable storage medium comprising instructions which, when executed by a computer, cause the computer to carry out the method of claim 18.
